(19) United States
(12) Plant Patent Application Publication
(10) Pub. No.: US 2012/0204300 P1
(43) Pub. Date: Aug. 9, 2012

(54) Variety of thunbergia plant named 'NEBTHU 007'

(50) ;

(75) Inventor: Hans de Does, Muenster (DE)

(73) Assignee: Volmary GmbH, Muenster (DE)

(21) Appl. No.: 12/931,597

(22) Filed: Feb. 4, 2011

(51) Int. Cl. A01H 5/00

(52) U.S. Cl. PLT/226

(57) ABSTRACT

‘NEBTHU 007’ is a new variety of thunbergia plant having dark velvet red/dark ochre flower colors and dark green foliage.

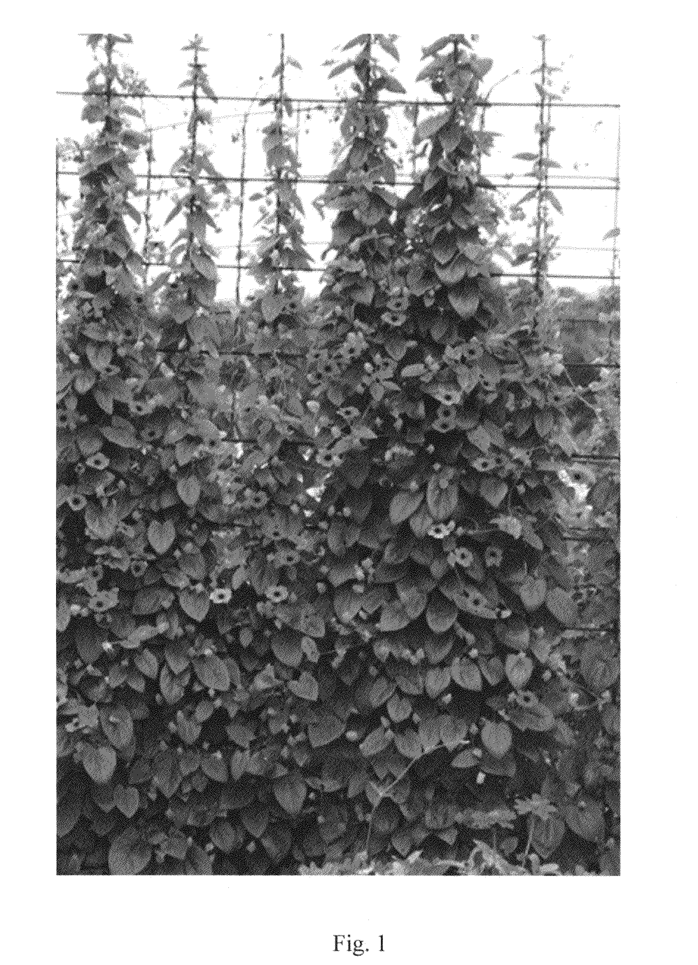

### BOTANICAL CLASSIFICATION

Thunbergia alata

### VARIETAL DENOMINATION

‘NEBTHU 007’

### BACKGROUND OF THE INVENTION

The present invention comprises a new and distinct cultivar of Thunbergia alata known by the varietal name ‘NEBTHU 007’. The new variety was discovered in Muenster, Germany in the Summer of 2007. The new variety is a result of a cross of proprietary and unpatented thunbergia plants. The purpose of the breeding program was to develop a thunbergia variety with improved flowers with new colors and deeper colored foliage. The new variety is similar in flower size to its female parent, but differs in flower color. ‘NEBTHU 007’ is similar in flower size and plant habit to its male parent, but differs in flower color and blooming time. Further, the new variety is similar in plant habit and flower size to thunbergia variety ‘African Sunset’ (unpatented), but differs in the colors of its corolla lobe, throat, and foliage. The new variety was first asexually reproduced by cuttings in September of 2007 in Muenster, Germany. The new variety has been trial and field tested and has been found to retain its distinctive characteristics and remain true to type through successive propagations.

The following characteristics have been repeatedly observed and are determined to be unique characteristics that distinguish ‘NEBTHU 007’ as a new and distinct cultivar from others known to the breeder:

1) Dark colored flowers that open velvet red and blush to dark ochre; and

2) Dark green foliage.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Muenster, Germany. The color readings were taken outside. The new variety was grown outside in the summertime under natural light at temperatures of 15-18° C. The new variety was 16 weeks old and grown in a 3 liter pot when described. Color references are primarily to the R.H.S. Colour Chart of The Royal Horticultural Society of London.
Plant:
   Time to initiate roots.—About 14 days at about 20° C.
   Time to develop roots.—About 30 days at about 18-20° C.
   Time to produce a finished flowering plant from a rooted cutting.—About 10 weeks in a 13 cm container.
   Rooting habit.—Shallow, with good branching.
   Form.—Climbing upward.
   Height from media surface to top of foliage.—100 cm, reaching a final garden height of over 2 meters.
   Height from media surface to top of flowers.—80 cm.
   Plant diameter.—35 cm.
   Vigor.—Medium to strong.
   Branching habit.—Poor to medium.
   Lateral branch.—Length: 10 cm after pinching. Diameter: 0.1 cm. Internode length: 10-12 cm. Strength: Weak. Texture: Light pubescence present. Color: Green.
   Foliage.—Arrangement: Single. Length 8 cm. Width: 4-5 cm. Shape of leaf: Spear-shaped. Apex: Acute. Base: Bow-shaped and concave. Margin: Spiny. Texture (both surfaces): Light pubescence present. Aspect: Flat, horizontal with a slight bend. Color: Young leaves: Upper surface: Green. Lower surface: Light gray-green. Mature leaves: Upper surface: Dark green. Lower surface: Gray-green. Petiole: Length: 3 cm. Diameter: 0.2 cm. Texture: Light pubescence present. Color: Green, later turning purple. Veins: Venation type: Feather. Color Upper surface: Green. Lower surface: Green — identical to foliage color.
Flowers:
   Flower arrangement.—Single.
   Flower habit.—Trumpet-like.
   Natural flowering season.—Spring until later summer (April until frost).
   Fragrance.—None present.
   Buds (described 1 day before opening).—Length: 2 cm. Diameter: 1.5 cm. Shape: Caudate. Color: Petals are cream with a purple base; calyx is green.
   Flowers.—Diameter: 3.5-4 cm. Height: 2.5-3 cm.
   Corolla.—Arrangement: Base is fused to the tube. Petals: Color: Upper surface (when opening): 179A. Upper surface (fully opened): 179A. Lower surface (when opening): 27A. Lower surface (fully opened): 27A. Shape: Truncate. Length: 3.5 cm. Width: 1.5-2 cm. Apex: Truncate and slightly unrelated. Base: Fused. Margin: Smooth and slightly unrelated. Number per whorl: 5. Texture: Smooth. Throat color: N77A. Tube color: N77A.
   Sepals.—Appearance: 2 spathes, like enveloping sepals. Length: 2.5 cm. Width: 2 cm. Apex: Obtuse. Base: Cordate. Margin: Entire. Texture: Smooth with light pubescence present. Color: Green.
   Peduncles.—Length: 3-3.5 cm. Width: 0.2 cm. Texture: Light pubescence present. Color: Green. Strength: Weak.
   Disease/pest resistance.—None observed to date.
   Temperature tolerance.—Medium.
   Drought tolerance.—Medium.
Reproductive organs:
   Stamens.—Number (per flower): 4. Anthers: Shape: Didynamous, bithecous. Length: 0.5 cm. Color: White. Pollen: Color: White-yellow. Amount: Low.
   Pistils.—Number (per flower): 1. Length: 1.7 cm. Style: Length: 1.5 cm. Color: White. Stigma: Shape: Bifid. Color: Cream-white. Size: 0.5 cm. Ovaries: Position: Superior. Length: 0.2 cm. Width: 0.3 cm. Color: Green.
   Seeds.—Number: 3-4. Length: 0.3-0.4 cm. Diameter: 0.3-0.4 cm. Color: Brown.

I claim:

1. A new and distinct variety of thunbergia plant named ‘NEBTHU 007’ as is herein described and illustrated.
